(19) United States
(12) Patent Application Publication
(10) Pub. No.: US 2014/0179532 A1
(43) Pub. Date: Jun. 26, 2014

(54) Oligourea Compounds and Method for Producing Same and Use Thereof

(71) Applicant: Werner Klockemann, Buchholz (DE)

(72) Inventor: Werner Klockemann, Buchholz (DE)

(21) Appl. No.: 13/985,283

(22) PCT Filed: Jan. 13, 2012
(86) PCT No.: PCT/DE2012/000025

§ 371 Date: Dec. 3, 2013

(30) Foreign Application Priority Data
Jan. 13, 2011 (DE) 102011008535.1

(51) Int. Cl. C07C 273/18; A01N 43/60

(52) U.S. Cl. CPC C07C 273/1809; A01N 43/60
USPC 504/155; 564/32; 544/400; 424/405; 514/252.12

(57) ABSTRACT

The invention relates to a method for producing oligourea compounds by chain building and depolymerization, and to oligourea dispersions, as well as to their use as fungicides, biocides and/or herbicides.

The subject matter of the invention is oligourea compounds, a method for their production, as well as their use.

The production of oligourea dispersions by depolymerization of polyurethane plastics, especially polyurethane flexible foams, is known and is described, e.g., in WO 2009/098226 A1. According to WO 2009/098226 A1, oligourea particles with particle size maxima between 100 and 1000 nm are obtained with a half-width of the particle size distribution of 100 to 10000 nm. In the depolymerization methods described thus far, oligourea particles are liberated that are formed during the foam reaction by reaction of water with the isocyanates. Therefore, one finds in the structure of these oligourea particles only the structure of normal urea (—NH—CO—NH—) among the isocyanate residues. The foam forming mechanism of polyurethane flexible foam is well understood and leads to the formation of so-called “urea balls” or “copolymer particles”, having a size of 200-500 nm (size determined by means of transmission electron microscope [TEM]) (Herrington R; and Hock K; Flexible Polyurethane Foams, 2^{nd} Ed., Midland, Mich., The Dow Chem Co: (1998). In the depolymerization methods described thus far, the oligourea particles formed in the foam structure (200-500 nm) are liberated and can be detected in the dispersions. The production of oligourea structures with amine ureas (e.g., —NH—R₁—CH₂—R₂—NH—) between the isocyanate residues, the production of smaller particles (e.g., 1-50 nm) and/or the production of products with narrower particle size distributions are not possible by these methods.

The problem of the invention is to provide urea compounds, optionally provided with reactive groups, especially dispersed in a dispersant, and a method for their production. A special problem of the present invention is to specifically produce the most uniform possible molecular particles, i.e., with narrow particle size distributions, in the nanometer range of under 40 nm in dispersion and without water.

It should be possible to provide the uniform molecular particles with functional, i.e., reactive groups, so that they can be processed into end products by reacting with another reaction component.

According to the invention, the problem is solved by the subject matter of the independent claims. Further embodiments, especially preferred embodiments, are the subject matter of the subclaims or specified below.

The subject matter of the invention is a method for the production of oligomeric urea compounds by reaction of starting compounds each with at least two reactive groups, chosen from hydroxy (—OH) and/or thiol (—SH—) groups, with di- or polyisocyanates at a first reaction temperature, in order to construct polyurethane and/or polythiourethane compounds, and depolymerization of the resulting polyurethane and/or polythiourethane compounds in the presence of a primary or secondary diamine or polyamine at a second reaction temperature, wherein the second reaction temperature, in regard to the maximum of the respective temperature, is at least 40° C., preferably at least 70° C., especially at least 100° C. higher than the first reaction temperature, in order to obtain compositions having oligomeric urea compounds that are present at least partly in particle form, and the starting compounds with at least two reactive groups, chosen from hydroxy (—OH) and/or thiol (—SH) groups. The starting compounds made accessible by depolymerization and any unreacted starting compounds act as diluents or dispersants.

The primary or secondary diamines or polyamines and/or additional monoamines are added preferably after formation of the polyurethane and/or polythiourethane compounds (partially in regard to the former), but before or during the temperature rise, especially before the temperature rise to the second reaction temperature.

This has the effect that free isocyanate groups that are preferably present after formation of the polyurethane and/or polythiourethane compounds, finish reacting with the amine, especially prior to the depolymerization. The depolymerization is then initiated by the temperature rise.

The depolymerization by raising the temperature brings about a cleavage of the urethane bond, which without going into the theory produces (again) free isocyanate bonds that react with the diamines or polyamines and any other monoamines present at the second reaction temperature.

According to one embodiment, dimer, trimer and tetramer oligomeric urea compounds are obtained, alongside monomeric urea compounds and oligomeric urea compounds with an oligomerization degree of 5 to 16. In particular, in terms of the oligourea molecules (number), possibly excluding the monomers, more than 50% and especially more than 80% of all oligourea molecules have oligomerization degrees of 2 to 16, especially 2 to 8. An oligomerization degree of 2 means that 2 monomer units are joined together, e.g., a diamine with an isocyanate.

The production of the polyurethane and/or polythiourethane compounds occurs preferably in the absence of other substances, such as are typically added in polyurethane reactions, like water, catalysts (tertiary amine and tin catalysts), colorants, stabilizers (like silicones) and/or inflators.

The polyurethane and/or polythiourethane compounds obtained as intermediate product are not present in solid form or do not go through any solid state, but instead are constantly present as liquid or in dissolved form during and under the conditions of the reaction.

According to one embodiment of the invention, a dispersant is added at least during the depolymerization. According to another variant, there is no adding of additional dispersant or solvent and the depolymerization occurs exclusively in the presence of the cleavage products as the dispersant or solvent.

The reactions can be carried out in customary agitator reactors, in dispersers, fast blenders, jet dispersers, reaction extruders, extruders or mixing kneaders. Preferably methods are used in which both reactions, chain construction, and depolymerization are done as a one-pot reaction in a single reactor.

When making the oligourea dispersions, in a first step a diisocyanate, preferably in molar excess, is brought into contact with a starting compound chosen as suitable for the eventual purpose of use with at least two groups, chosen from —OH and/or —SH, and toward the end of the reaction of the diisocyanate with the starting compound an amine or an amine mixture having primary and/or secondary amine groups is added to the prepolymer and brought into a reaction, and urea bonds or urea compounds are formed by the cleavage of the polyurethane/polythiourethane bond brought about by temperature rise, whose size is determined by the molar ratio of isocyanate to the starting compound and the type of the amine. The urea compounds are dispersed as nanoscale particles in the liberated polyhydroxy/polythio compound and the optional dispersant.

The reaction to form the polyurethane or polythiourethane compound occurs, e.g., for 10 minutes to 8 hours and depending on this at temperatures between 20 and 120° C. The liquid prepolymer immediately thereafter or after any desired time is brought into contact with an amine compound, possibly in the presence of a dispersant and under conditions where the isocyanate group reacted with the amine groups of the amine compound and the urethane/thiourethane groups (after cleavage) are cloven at least partly or also completely with the amine groups to form urea groups, under liberation of the starting compound used during the synthesis of the prepolymer at temperatures of 120 to 250° C., especially 120 to 220° C., especially 120 to 180° C. The urea compounds can be dispersed as nanoscale particles in the dispersant.

The dispersants used according to the invention or additional ones can preferably be diols, including polyether alcohols. The diols can be simple diols, such as ethylene glycol, diethylene glycol, triethylene glycol, tetraethylene glycol, higher polyethylene glycols, propylene glycol, dipropylene glycol, tripropylene glycol, tetrapropylene glycol, higher propylene glycols, butane-1,4-diol, α,ω-bishydroxy-butylene glycols, copolymers of ethylene oxide and propylene oxide, wherein the long-chain diols can have molecular weights up to 6000.

The polyether alcohols can be difunctional or polyfunctional, in general the typical polyether alcohols of polyurethane chemistry will be used, e.g., polyether triols with glycerol or trimethylol propane as starter and propylene oxide and possibly ethylene oxide statistically distributed or as inner or terminal block, wherein the molecular weight is between 400 and 6000.

The additionally used dispersant, if it has reactive groups for the isocyanate group, is added first to the depolymerization reaction. Essentially other compounds are also suitable as dispersant if they do not dissolve the oligourea molecules, such as inert compounds without reactive groups.

Amines or amine mixtures which can be used according to the invention are
a) compounds having in total at least two primary, two secondary, or at least one primary and at least one secondary amine group

and optionally in addition
b) primary or secondary monoamines which can possibly have other functional groups, such as —OH, —SH, which do not react under the conditions of the cleavage
c) ammonia and optionally water.

Suitable amines of group a) are, e.g., urea, hexane-1,6-diamine, di-iso-propylamine, ethylene diamine, N,N′-dimethyl-ethylene diamine, 1,3-propylene diamine, isophorone-diamine, 4,4′-diaminodicyclohexylmethane, diethylene triamine, triethylene tetramine, N,N-bis-(2-aminopropyl)methylamine, N,N-bis-(3-aminopropyl)methylamine, dipropylene triamine, tripropylene tetramine, 1,4-phenylene diamine, guanidine, poly-guadine, 1,3-phenylene diamine, 4,4′-diaminodiphenylmethane, triamine nonane, and so on. Furthermore compounds containing amino groups are suitable, including polymers such as α,ω-diaminopolyether, Mannich bases or oligoethylene imines. In particular, α,ω-diaminopolyethers based on bis-hydroxypropylene glycols of molecular weight 200 to 2000 can be used.

In the sense of the present invention, urea is regarded as a compound with two primary amine groups and assigned to group a).

Suitable amines of group b) are di-n-butylamine or di-iso-butylamine. For the production of hydroxyl-functional nanoscale ureas in functional dispersants, alkanolamines can be added to the functional urea nanodispersions of the invention as chain interruptors, in which the amino group reacts with the isocyanates to form urea groups and the less reactive hydroxyl group remains freely available (at least in the presence of amines). In this way, hydroxyl-functional urea nanodispersions are produced. Suitable alkanolamines are, e.g., ethanolamine, diethanolamine, N-methylethanolamine, 2- or 3-propanolamine, dipropanolamine, N-methyl-propanolamine, etc.

Other suitable compounds that can be introduced by means of one or more amine groups are compounds containing at least one primary or secondary amine group and furthermore having a phosphate group (e.g., for use as flame retardant) or compounds containing at least one primary or secondary amine group and furthermore having a sulfur group (e.g., for use as a fungicide or biocide). An example of a compound with two primary amine groups is guanidine (the ═NH group is little reactive if at all in the context of the depolymerization reaction), an example with two secondary amine groups is 2-methylthio-4-tert-butylamino-6-cyclopropylamino-s-triazine (CAS: 28159-98-0, commercial names: Irgarol 1051 or Cybutryn). In this case, the group acting as a biocide is built into the chain and not only at the end of the chain.

Thus, oligomers with persistent biocidal properties can be produced by reacting one or more biocidally active compounds each with at least two chemically reactive groups of oligoureas, e.g., in the molar ratio of 1:1 to 1:4, and optionally additional chain lengtheners, spacers, and/or crosslinkers, with at least three reactive groups. The reaction produces polymers with persistent biocidal properties that have structural units of one or more biocidally active compounds with at least two chemically reactive groups and a second long-chain unit with at least two groups that are reactive and reacted with the reactive groups and optionally other chain lengtheners, spacers, and/or crosslinkers with at least three reactive groups.

Surprisingly, it was found that certain biocide compounds also retain their biocidal effect when they are incorporated into an oligomer's main chain. Thus, for example, 2-methylthio-4-tert-butylamino-6-cyclopropyl-amino-s-triazine, known as Irgarol® 1051, can react by the two NH groups with di- and/or polyisocyanates or with prepolymers based on them and yield, depending on the structure of the isocyanate component and the polyol component, elastic, semihard or hard poly(urethane ureas) that are all biocidal to algae, bacteria and microfungi (fungi) due to the incorporation of the compound. 2-methylthio-4-tert-butylamino-6-cyclopropylamino-s-triazine is only one example of a biocidally active molecule that is also a highly effective building block in the polyaddition process with di- and/or polyisocyanates, besides [having] a biocidal action.

Other biocidally active compounds that can be used in the method of the invention for the production of persistent biocidally active polymers according to the invention are carbendazim [(N-(benzimidazol-2-yl)carbamidic acid methyl ester], N-(3-aminopropyl)-N-dodecylpropan-1,6-diamine, N-dodecyl-propylene diamine, 5-chlor-2-methyl-isothiazolinone, 2-octyl-3(2H)-isothiazolinone, methyloxazolidine, dichloroctylisothiazolinone (Vinyzene DCOIT), octylisothiazolinone, N,N′-diethylpiperazine, N,N′-dibenzylpiperazine, N,N′-dicyclohexyl-piperazine, piperidine, as well as other substituted oxazolidines, benzimidazole, piperazine, piperidine or isothiazoline. The incorporated quantity of biocidally active compounds according to the invention is between 0.01 and 10 wt. % of the composition thus obtained.

Suitable di- or polyisocyanates are preferably all familiar aliphatic, cycloaliphatic, araliphatic or aromatic di- or polyisocyanates. Examples are 4,4′-diphenylmethane diisocyanate, 1,4-phenylene-diisocyanaet, 1,4-xylylene diisocyanate, toluoylene-1,4-diisocyanate, toluoylene-1,6-diisocyanate, 2,4′-diphenylmethane diisocyanate, 2,2′-diphenylmethane diisocyanate, hexane-1,6-diisocyanate, iso-phorone diisocyanate, tetramethylxylylene diisocyanate, 4,4′-dicyclohexyl-methane diisocyanate, triisocyanatononane, cyclohexane-1,4-diisocyanate and so on.

The di- or polyhydroxy compound is in particular a diol, but it can also be a triol or tetrol, for example, or mixtures of these. Polyether alcohols are preferred as the polyhydroxy compound.

Besides or instead of the amines used to complete the reaction of the excess isocyanate groups by the chain building reaction, water and/or ammonia can also be used.

The oligourea dispersions according to the invention contain a dispersant or diluent, and have
oligourea molecules with 2 to 16, preferably 2 to 8 monomer units, especially on average (possibly excluding monomers) with 2 to 16, preferably on average 2 to 8, monomer units,
   wherein especially more than 50%, especially more than 80%, of all urea/oligourea molecules (possibly excluding the monomers) have oligomerization degrees of 2 to 16, especially 2 to 8, especially preferably 2 to 6.

The oligourea molecules in dispersion can be characterized especially by one or more of the following features:
a) the oligourea molecules form in the dispersant particles sizes on average of 4 to 40 nm, preferably 8 to 20 nm.
b) the oligourea molecules form in the dispersant particle sizes of less than 40 nm for 90% of all particles, especially less than 30 nm for 90% of all particles and especially preferably less than 20 nm for 90% of all particles (each time measured by laser light scattering (Zetasizer S, Malvern) and/or.
c) the oligourea molecules form in the dispersant in particular particle size distributions such that 80% of all particles, especially 90% of all particles have a particle size of minus 10 to plus 10 nm of the mean value.

In particular, the oligourea compounds are present as particles in dispersion. According to another alternative, they are present entirely or partly in solution.

The terminal groups can be at least one free amino and/or hydroxyl group, carboxyl, or SH group (e.g., two amino groups, two hydroxyl groups, or an amino and a hydroxyl group) as the terminal group.

The indicated nanometers pertain each time to the particle diameter according to the hydrodynamic volume and as is determined by laser light scattering (Zetasizer S, Malvern) in the respective dispersion.

The dispersant can likewise have functional groups, preferably two free/functional groups per molecule, chosen from the group —OH, —NH₂, ═NH, preferably entirely or partly —OH.

The functional groups of the oligourea particles and the dispersant(s) are preferably chosen such that they do not react with each other in the functional urea nanodispersion, at least after completion of the reaction.

In particular, the oligourea dispersions of the invention contain or consist of:
1 to 90 wt. %, or 5 to 50 wt. % of mono- and/or oligourea molecules,
99 to 10 wt. %, or 95 to 50 wt. %, of a dispersant, which has at least one —OH and/or —SH group.

The mono- and oligourea molecules used according to the invention consist, for example, of the base bodies of suitable di- and/or polyisocyanates that have been reacted with mono-, di- and/or polyamines and accordingly are present as di- and/or trisubstituted ureas, wherein preferably one terminal group at the ureas is a primary or secondary amino group.

These urea molecules can generally be represented in relation to the reaction with a diisocyanate and a diamine as:

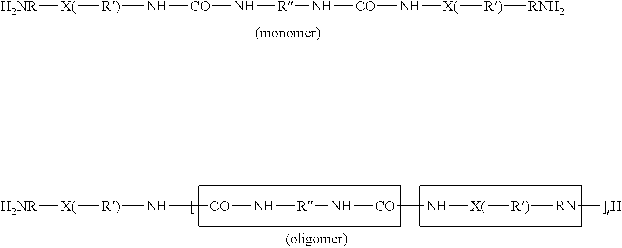

where
R′: hydrogen, an aliphatic or aromatic residue,
X: a —CH₂—, —CH═, —C₂H₄—, —C₃H₈— or higher aliphatic group, a —CₙHₙ₊₂NH— group, a —CₙHₙ₊₂N(CₙHₙ₊2)ₘNH— group, and
R″: an aliphatic C₄— to C₁₆— residue, an aromatic residue, a biphenyl residue, a diphenylmethane residue.
n: 1 to 18 and m=1 or 2 and
r: 2 to 20.

The invention will be explained by the following experimental examples, without being confined to them.

### EXAMPLES

### Analytics

The particle size was determined by means of laser light scattering (Nanophox® of Sympatec GmbH (PCCS), Zetasizer S, Malvern GmbH) in dispersion, in the medium resulting each time from the reaction. The maximum of the distribution curve is indicated in nm (diameter per hydrodynamic volume).

The measurement of the particle sizes was done by 2 different methods on 2 instrumental systems: Zetasizer S (Malvern Co.) and Nanophox (Sympatec Co.). Both instruments use the principle of dynamic light scattering for the particle size determination.

In instrumental systems that measure by means of dynamic light scattering, a measurement is taken by sending a laser beam through a specimen. Light scattering produces on the detector window an interference pattern of bright and dark spots. Small particles have a higher mobility than large particles and cause a faster fluctuating bright-dark condition on the monitor screen over a particular period of time. From the speed of fluctuation of the bright spots, one can infer the size of the particles (Stokes-Einstein relation).

In the Nanophox instrument system, the Photon Cross Correlation Spectroscopy (PCCS) technique is used for the measurement and evaluation. PCCS is a technique allowing one to take measurements at the same time of particle size and stability in the nanometer to micrometer range in suspension and emulsions. The key principle of PCCS is a 3D cross correlation technique. Thanks to a special scatter geometry, the cross correlation of the scattered light is able to separate precisely the fraction of single scattered light from that of multiple scattered light. The instrument works with two separate laser beams and two detectors.

The particle size distribution as determined with the Zetasizer S is shown as a graph (intensity in % versus particle diameter in nm). There are shown:

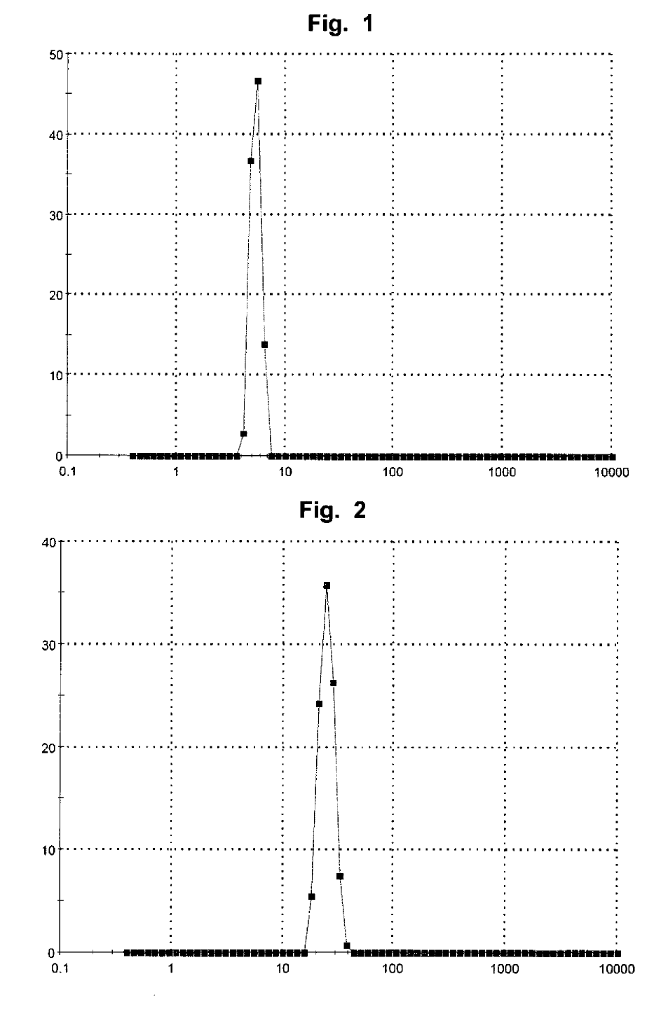

FIG. 1 The particle size distribution (diameter) of the particles in the composition of example 6 as measured in the dispersion obtained there

FIG. 2 The particle size distribution (diameter) of the particles in the composition of example 7 as measured in the dispersion obtained there

Both measurements are taken in the dispersant at 40° as was present after the reaction. The size of MDA is assumed to be 1.8 nm A product of 1 molecule of MDI and 2 molecules of DETA was calculated at 2.5 nm. Thus, in theory, a measured particle size of 10 nm could be a tetramer of this structure, 20 nm particles could in theory be an octamer of this structure, and so on. The viscosity was determined as the kinematic viscosity by rotation each time at 25° C. and according to DIN 53019 (instrument: Rheostress 300). The isocyanate content was determined by DIN 53185, the OH number by DIN 53240 and the amine number by DIN 16945.

Educts used

| DMD: | 4,4′-diphenylmethane diisocyanate in flake form |
|---|---|
| Lupranol ® | polypropylene glycol (diol) with a mean molecular |
| 1100: | weight of 1100 g/mol, OH number 104, Elastogran AG |
| Lupranol ® | polypropylene glycol (diol) with a mean molecular |
| 1000: | weight of 2000 g/mol, OH number 55, Elastogran AG |
| DPG: | dipropylene glycol |
| DEG: | diethylene glycol |
| DETA: | diethylene triamine |
| DBA: | di-n-butylamine |
| DPTA: | dipropylene triamine |
| PTMO 1000: | poly(tetramethylene oxid)diol with mean molecular |
| | weight of 1000 g/mol |
| BD14: | butane-1,4-diol |
| TCD: | tricyclo-diamine-decane-diamine |
| PC-Amin ® | bis-N,N-(2-aminopropyl)methylamine, Performance |
| DA145: | Chemicals GmbH |
| Lupranol ® | polypropylene glycol on a sugar base with a mean |
| 3403: | molecular weight of 550 g/mol, OH number: 445, |
| | Elastogran AG |
| Lupranol ® | polypropylene glycol with a mean molecular weight of |
| 1200: | 450 g/mol, diol, OH number: 248, Elastogran AG |
| PolyTHF | polytetrahydrofuran with 2 hydroxy grops and a mean |
| 650 S: | molecular weight of 650 g/mol, BASF AG |
| Vorastar | Dow Chemicals prepolymer with residual NCO of 15.8%, |
| HB 6549: | polymer chain polycarprolactam |
| NAEP: | N-aminoethylpiperazine |
| Lupranol ® | polypropylene glycol (triol) with a mean molecular weight |
| 2032: | of 3100 g/mol, OH number 55, Elastogran AG |

### SAMPLE EMBODIMENTS

### Example 1

Synthesis of the precursor (first stage): in a 10 1 double-wall refined steel reactor with agitator, heating by thermal oil, dispensers for liquids and solids, nitrogen introduction and heat exchanger, 2.5 kg of DMD was placed, heated to 45° C. and melted. To the liquid isocyanate was slowly added 5.5 kg of Lupranol® 1100 while stifling, so that the temperature did not exceed 55° C. The mixture was then further stirred at 46° C. for another hour. The result was a homogeneous, viscous, yellowish product with isocyanate content of 5.32%.

Reaction of the precursor (second stage): In a 20 1 double-wall refined steel reactor with agitator, heating by thermal oil, dispensers for liquids and solids, nitrogen introduction and heat exchanger, 3.2 kg of DPG, 0.8 kg of DETA and 0.5 kg of DBA were measured out and this mixture was heated under stifling to 160° C. In the space of 40 minutes, 5.5 kg of the above prepared precursor was added.

After the adding was complete, the mixture was stirred at 180° C. for an additional 45 min. The reaction mixture was let out through a bottom valve. It was homogeneous, easily flowing, brown-orange and clear. The nanodispersion contained 31.4 wt. % of oligoureas with amine terminal groups. Determination of the particle size of amine-functional oligoureas of the nanodispersion by means of Nanophox® (Sympatec GmbH, PCCS) and Zetasizer S (Malvern) revealed the maximum of the particle size distribution curve (diameter per hydrodynam. vol.) at 12 nm and a distribution of 10 to 14 nm. The nanodispersion had a hydroxyl number of 410 mg KOH/g, an amine number of 92 mg KOH/g and a viscosity (rotation) of 720 mPas (25° C.).

### Example 2

The method described in example 1 was carried out as a continuous method in a reaction extruder. At first, the synthesis of the precursor was done under uniform dispensing and mixing of the melted diisocyanate and the diol (molar ratio 2:1) with 5.0 kg of 4,4′-DMD and 11.0 kg of Lupranol® 1100) per hour at 45° C. to 70° C. (temperature gradient) in the front part of the extruder. The reaction of the resulting precursor occurred directly thereafter in the second part of the extruder (heating zones 4 to 8) at 180° C. under dispensing of the premixed solvolysis mixture (32 parts of DPG, 8 parts of DETA and 5 parts of DBA). The product was homogeneous, easily flowing, brown-orange and clear. The nanodispersion contained 31.4 wt. % of oligoureas. Determination of the particle size of amine-functional oligoureas in the nanodispersion by means of Nanophox® (Sympatec GmbH, PCCS) and Zetasizer S (Malvern) revealed the maximum of the particle size distribution curve at 12 nm and a distribution of 10 to 14 nm. The nanodispersion had a hydroxyl number of 425 mg KOH/g, an amine number of 95 mg KOH/g and a viscosity (rotation) of 720 mPas (25° C.).

### Example 3

Synthesis of the precursor (first stage): in a 10 1 refined steel reactor with agitator, heating by thermal oil, dispensers for liquids and solids, nitrogen introduction and heat exchanger, 2.7 kg of DMD was heated to 45° C. and to this was added slowly under stirring 5 kg of PTMO 1000 (around 1.5 hours), so that the temperature did not rise above 75° C. The mixture was then stirred at 60° C. for around 1 h. There was obtained a homogeneous, viscous, yellowish product with isocyanate content of 5.5%.

Reaction of the precursor (second stage): 5.5 kg of this precursor was added via a bottom drain line directly into a 20 1 refined steel reactor with agitator, heating by thermal oil, nitrogen introduction and heat exchanger with a mixture of 3.2 kg of DPG, 0.9 kg of DPTA and 0.5 kg of DBA heated to 120° C. and after this mixture was completely added it was heated under stirring to 180° C. Further stirring was done at 180° C. for 30 minutes, after which the reaction mixture was drained by means of a bottom valve. It was homogeneous, flowing and clear. The nanodispersion contained 19.2 wt. % of amine-functional oligoureas. Determination of the particle size of amine-functional oligoureas in the nanodispersion by means of Zetasizer (Malvern) revealed the maximum of the particle size distribution curve at 11 nm. The nanodispersion had a hydroxyl number of 340 mg KOH/g, an amine number of 92 mg KOH/g and a viscosity (rotation) of 400 mPas (25° C.).

### Example 4

Synthesis of the precursor (first stage): in a 10 1 double-wall refined steel reactor with agitator, heating by thermal oil, dispensers for liquids and solids, nitrogen introduction and heat exchanger, 5 kg of DMD was placed, heated to 45° C. and melted. To the liquid isocyanate was slowly added 4.5 kg of Lupranol® 1200 while stirring, so that the temperature did not exceed 55° C. The mixture was then further stirred at 46° C. for another hour. The result was a homogeneous, viscous, yellowish product with isocyanate content of 6.8%.

Reaction of the precursor (second stage): In a 20 1 refined steel reactor with agitator, heating by thermal oil, dispensers for liquids and solids, nitrogen introduction and heat exchanger, 2.1 kg of DPG, 1.5 kg of DETA and 0.4 kg of DBA and 1 kg of BD14 were measured out and this mixture was heated under stirring to 160° C. In the space of 40 minutes, 5 kg of the above prepared precursor was added. After the adding was complete, the mixture was stirred at 180° C. for an additional 45 min. The reaction mixture was let out through a bottom valve. It was homogeneous, easily flowing, yellow and clear. The nanodispersion contained around 50% of oligoureas with amine terminal groups. Determination of the particle size of amine-functional oligoureas in the nanodispersion by means of Zetasizer (Malvern) revealed the maximum of the particle size distribution curve at 3.5 nm and a distribution of 2 to 6 nm. The nanodispersion had a hydroxyl number of 568 mg KOH/g, an amine number of 187 mg KOH/g and a viscosity (rotation) of 909 mPas (25° C.).

### Example 5

Synthesis of the precursor (first stage): in a 10 1 refined steel reactor with agitator, heating by thermal oil, dispensers for liquids and solids, nitrogen introduction and heat exchanger, 3.0 kg of DMD was heated to 45° C. and thus melted and this was slowly mixed under stifling with 3.9 kg of PolyTHF 650 S, so that the reaction temperature did not rise above 60° C. The mixture was then stirred at 55° C. for around 1 h. There was obtained a homogeneous, viscous, yellowish, slightly cloudy product with an isocyanate content of 7.0%.

Reaction of the precursor (second stage): 2.0 kg of DPG, 1.2 kg of DEG, 0.9 kg of PC-Amin® DA145 and 0.5 kg of DBA was measured out into a 20 1 refined steel reactor with agitator, heating by thermal oil, dispensers for liquids and solids, nitrogen introduction and heat exchanger and this mixture was heated under stirring to 160° C. In the space of 60 minutes, 5.5 kg of the above prepared precursor was added. After the adding was complete, the mixture was stirred at 180° C. for an additional 30 min. The reaction mixture was let out through a bottom valve. It was homogeneous, easily flowing, and clear at 35° C. The nanodispersion contained around 24.2 wt. % of amine-functional oligoureas. Determination of the particle size of amine-functional oligoureas in the nanodispersion by means of Zetasizer (Malvern) revealed the maximum of the particle size distribution curve at 5.8 nm. The nanodispersion had a hydroxyl number of 422 mg KOH/g, an amine number of 127 mg KOH/g and a viscosity (rotation) of 590 mPas (25° C.).

### Example 6

Synthesis of the precursor (first stage): in a 20 1 refined steel reactor with agitator, heating by thermal oil, dispensers for liquids and solids, nitrogen introduction and heat exchanger, 5 kg of DMD was placed, heated to 45° C. and melted. To the liquid isocyanate was slowly added 6.5 kg of Poly-THF 650S while stifling, so that the temperature did not exceed 55° C. The mixture was then further stirred at 46° C. for another hour. The result was a homogeneous, viscous, yellowish product with isocyanate content of 7%.

Reaction of the precursor (second stage): 3.8 kg of DPG, 0.7 kg of DETA and 1.2 kg of Lupranol 2032 was measured out into a 20 1 refined steel reactor with agitator, heating by thermal oil, dispensers for liquids and solids, nitrogen introduction and heat exchanger and this mixture was heated under stifling to 160° C. In the space of 40 minutes, 4.3 kg of the above prepared precursor was added. After the adding was complete, the mixture was stirred at 180° C. for an additional 45 min. The reaction mixture was let out through a bottom valve. It was homogeneous, easily flowing, yellow and clear. The nanodispersion contained 31.4 wt. % of oligoureas with amine terminal groups. Determination of the particle size of amine-functional oligoureas in the nanodispersion by means of Zetasizer (Malvern) revealed the maximum of the particle size distribution curve at 5.4 nm and a distribution of 4 to 8 nm. The particle size distribution curve is shown in FIG. 1. The nanodispersion had a hydroxyl number of 416 mg KOH/g, an amine number of 74 mg KOH/g and a viscosity (rotation) of 1690 mPas (25° C.).

### Example 7

Synthesis of the precursor (first stage): in a 20 1 double-wall refined steel reactor with agitator, heating by thermal oil, dispensers for liquids and solids, nitrogen introduction and heat exchanger, 5 kg of DMD was placed, heated to 45° C. and melted. To the liquid isocyanate was slowly added 6.5 kg of Poly-THF 650S (MG 650) while stifling, so that the temperature did not exceed 55° C. The mixture was then further stifled at 46° C. for another hour. The result was a homogeneous, viscous, yellowish product with isocyanate content of 7%.

Reaction of the precursor (second stage): into a 20 1 refined steel reactor with agitator, heating by thermal oil, dispensers for liquids and solids, nitrogen introduction and heat exchanger was measured out 3.7 kg of dipropylene glycol, 1 kg of diethylene triamine 0.4 kg of dibutylamine and 0.9 kg of polypropylene glycol MG 3100 (e.g., Lupranol 2032, Elastogran AG) and this mixture was heated while stirring to 160° C. In the space of 40 minutes, 4 kg of the above prepared precursor was added. After the adding was complete, the mixture was stifled at 180° C. for an additional 45 min. The reaction mixture was let out through a bottom valve. It was homogeneous, easily flowing, yellow and clear. The nanodispersion contained around 27% of oligoureas with amine terminal groups. Determination of the particle size of amine-functional oligoureas in the nanodispersion by means of Zetasizer (Malvern) revealed the maximum of the distribution curve at 25 nm and the distribution of 14 to 35 nm. The particle size distribution curve is shown in FIG. 2. The nanodispersion had a hydroxyl number of 445 mg KOH/g, an amine number of 115 mg KOH/g and a viscosity (rotation) of 1090 mPas (25° C.).

### Example 8

Synthesis of a nanodispersion containing amino-functional mono- and bifunctional mono- and oligoureas from 4,4′-MDI and N-aminoethylpiperazine (NAEP) and diethyltriamine (DETA), in a refined steel reactor (two-stage method).

Synthesis of the precursor (first stage): in a 10 1 double-wall refined steel reactor with agitator, heating by thermal oil, dispensers for liquids and solids, nitrogen introduction and heat exchanger, 2.5 kg of DMD was placed, heated to 45° C. and melted. To the liquid isocyanate was slowly added 5.5 kg of Lupranol® 1100 while stifling, so that the temperature did not exceed 55° C. The mixture was then further stirred at 46° C. for another hour. The result was a homogeneous, viscous, yellowish product with isocyanate content of 5.32%.

Reaction of the precursor (second stage): into a 20 1 refined steel reactor with agitator, heating by thermal oil, dispensers for liquids and solids, nitrogen introduction and heat exchanger was measured out 4.14 kg of dipropylene glycol, 0.37 kg of diethylene triamine and 0.67 kg of aminoethylpiperazine (NAEP) and this mixture was heated while stirring to 160° C. In the space of 40 minutes, 4.82 kg of the above prepared precursor was added. After the adding was complete, the mixture was stifled at 180° C. for an additional 45 min. The reaction mixture was let out through a bottom valve. It was homogeneous, easily flowing, brown-orange and clear. The nanodispersion contained around 24% of oligoureas with amine terminal groups. Determination of the particle size of amine-functional oligoureas in the nanodispersion by means of Zetasizer (Malvern) revealed the maximum of the particle size distribution curve at 5.2 nm and a distribution of 3 to 8 nm. The nanodispersion had a hydroxyl number of 434 mg KOH/g, an amine number of 102 mg KOH/g and a viscosity (rotation) of 1860 mPas (25° C.). The nanodispersion has biocidal action against algae, daphnia and bacteria. It can be used to make coatings with biocidal action.

### Example 9

One-stage method: in a 20 1 double-wall refined steel reactor with agitator, heating by thermal oil, dispensers for liquids and solids, nitrogen introduction and heat exchanger, 1.5 kg of DMD was placed, heated to 45° C. and melted. To the liquid isocyanate was added 3.44 kg of Lupranol® 1100, 4.3 kg of DPG and 0.7 kg of DETA while stirring. At the same time, the mixture was heated under stirring to 180° C. and stirred at this temperature for 30 minutes. The reaction mixture was let out through a bottom valve. It was homogeneous, easily flowing, yellow and clear. The nanodispersion contained around 22.6% of oligoureas with amine terminal groups. Determination of the particle size of amine-functional oligoureas in the nanodispersion by means of Nanophox® (Sympatec GmbH, PCCS) and Zetasizer (Malvern) revealed the maximum of the particle size distribution curve at 12 nm and the distribution of 8 to 15 nm. The nanodispersion had a hydroxyl number of 457 mg KOH/g, an amine number of 78 mg KOH/g and a viscosity (rotation) of 851 mPas (25° C.). The nanodispersion can be used for production of coatings.

### Example 10

Synthesis of the precursor (first stage): in a 10 1 double-wall refined steel reactor with agitator, heating by thermal oil, dispensers for liquids and solids, nitrogen introduction and heat exchanger, 2.5 kg of DMD was placed, heated to 45° C. and melted. To the liquid isocyanate was slowly added 5.5 kg of Lupranol® 1100 while stifling, so that the temperature did not exceed 55° C. The mixture was then further stirred at 46° C. for another hour. The result was a homogeneous, viscous, yellowish product with isocyanate content of 5.32%.

Reaction of the precursor (second stage): into a 20 1 refined steel reactor with agitator, heating by thermal oil, dispensers for liquids and solids, nitrogen introduction and heat exchanger was measured out 3.95 kg of DPG, 0.83 kg of DETA and 0.64 kg of TCD and this mixture was heated while stirring to 160° C. In the space of 20 minutes, 4.58 kg of the above prepared precursor was added. After the adding was complete, the mixture was stifled at 180° C. for an additional 30 min. The reaction mixture was let out through a bottom valve. It was homogeneous, easily flowing, yellow and clear. The nanodispersion contained around 30% of oligoureas with amine terminal groups. Determination of the particle size of amine-functional oligoureas in the nanodispersion by means of Zetasizer (Malvern) revealed the maximum of the particle size distribution curve at 4.7 nm and a distribution of 3 to 8 nm. The nanodispersion had a hydroxyl number of 496 mg KOH/g, an amine number of 143 mg KOH/g and a viscosity (rotation) of 707 mPas (25° C.).

### Example 11

In a 250 ml sulfurating flask (one-step method, intensive condenser, nitrogen supply and temperature sensor, magnetic agitator) 15.6 g of DMD was melted (47° C.) under nitrogen gas supply and then a mixture of 4 g of DETA, 6 g of NAEP, 34.4 g of Lupranol 1100 and 40 g of DPG was added and stirred under temperature rise to 180° C. The mixture at first solidified and after 30 min of reaction time it yielded an optically clear product with an oligourea content of around 23 wt. %, an OH number of 437, an amine number of 86 and a viscosity (rotation) of 1850 mPas (25° C.). The nanodispersion can be used to produce coatings.

### Example 12

Synthesis of a nanodispersion containing amino-functional mono- and oligoureas from Vorastar HB 6549, DETA in the sulfurating flask (single-step method). In a 250 ml sulfurating flask (intensive condenser, nitrogen supply and temperature sensor, magnetic agitator) 41.1 g of DETA was heated under stifling to 160° C. and 58.9 g of Vorastar HB 6549 was added under stirring. After 60 min of reaction time at 180° C. there resulted a yellow, homogeneous, slightly cloudy product containing around 50% oligoureas, with a viscosity of 7420 mPas (25° C.), an OH number of 548 and an amine number of 428. The nanodispersion can be used to produce coatings.

### Example 13

In a 250 ml sulfurating flask (two-step method, intensive condenser, nitrogen supply and temperature sensor, magnetic agitator) 50 g of DPG and 10 g of DETA was heated to 180° C. under stirring. To the mixture was added 10 g of a polymer of trimerized HDI and a tetrafunctional SH-functional compound (pentaerythritol-tetra(3-mercaptopropionate), THIOCURE® PETMP, BRUNO BOCK Chemische Fabrik GmbH & Co. KG). The mixture was heated to 210° C. and stirred for 1 hour. After 60 minutes of reaction time, a brown, optically clear, homogeneous product resulted with a theoretical oligourea content of 12% and a viscosity of 864 mPas. The nanodispersion can be used to produce coatings.

### Example 14

In a 250 ml sulfurating flask (two-step method, intensive condenser, nitrogen supply and temperature sensor, magnetic agitator) 20 g of DETA was heated to 180° C. under stirring. To the DETA was added 15.27 g of a polymer of trimerized HDI (Desomdur N 3900) and a tetrafunctional SH-functional compound (pentaerythritol-tetra(3-mercaptopropionate), THIOCURE® PETMP, BRUNO BOCK Chemische Fabrik GmbH & Co. KG). The mixture was heated to 205° C. and stirred for 1.5 hour. After the reaction time, a brown, optically clear, homogeneous product resulted with a theoretical oligourea content of 36.8% and a viscosity of 1500 mPas. The nanodispersion can be used to produce coatings.

### Example 15

In a 250 ml sulfurating flask (two-step method, intensive condenser, nitrogen supply and temperature sensor, magnetic agitator) 50 g of DPG and 10 g of adipic acid was heated to 180° C. under stifling. To the mixture was added 10 g of a polymer of trimerized HDI and a tetrafunctional SH-functional compound. The mixture was heated to 210° C. and stirred for 2 hours. After the reaction time, a yellow, optically clear, homogeneous product resulted with a viscosity of 820 mPas. The nanodispersion can be used to produce coatings.

### Example 16 (Two-Phase)

Synthesis of the prepolymer: in a 10 1 refined steel reactor with agitator, heating by thermal oil, dispensers for liquids and solids, nitrogen introduction and heat exchanger, 2.5 kg of DMD was heated to 45° C. and thereby melted, and this was mixed slowly with 10 kg of Lupranol® 1000 under stifling. The mixture was then further stirred at 46° C. for another hour. The result was a homogeneous, viscous, yellowish, slightly cloudy prepolymer with an isocyanate content of 4.13%.

Reaction of the prepolymer: 3.2 kg of DPG, 0.8 kg of DETA and 0.5 kg of DBA was measured out into a 12.5 1 refined steel reactor with agitator, heating by thermal oil, dispensers for liquids and solids, nitrogen introduction and heat exchanger and this mixture was heated under stifling to 160° C. In the space of 60 minutes, 5.5 kg of the above prepared prepolymer was added.

After the adding was complete, the mixture was stirred at 180° C. for an additional 30 minutes. The reaction mixture was let out through a bottom valve. It is two-phase after standing for 24 h at room temperature. The dispersion (clear lower phase) was homogeneous, flowing, and clear at 65° C. The dispersion contained 20 wt. % of amine-functional urea particles. Determination of the particle size of the amine-functional oligoureas in the reaction sol by means of Nanophox® (Sympatec GmbH, PCCS) revealed the maximum of the distribution curve at 11 nm and the distribution of 9 to 15 nm. The nanodispersion had a hydroxyl number of 653 mg KOH/g, an amine number of 198 mg KOH/g and a viscosity (rotation) of 390 mPas (25° C.).

1. Method for the production of oligomeric urea compounds by reaction of starting compounds each with at least two reactive groups, chosen from the hydroxy and/or thiol group, with di- or polyisocyanates
at a first reaction temperature, in order to construct polyurethane and/or polythiourethane compounds (chain construction),
and subsequent depolymerization of the resulting polyurethane and/or polythiourethane compounds in the presence of a primary or secondary diamine or polyamine at a second reaction temperature, wherein the second reaction temperature, in regard to the maximum of the respective temperature, is at least 40° C. higher than the first reaction temperature, in order to obtain compositions having oligomeric urea compounds and the starting compounds with at least two reactive groups, chosen from the hydroxy and/or thiol group.

2. Method according to claim 1, wherein compositions are obtained having oligomeric urea compounds with an oligomerization degree of 2 to 16 and independently of this, in terms of the oligourea molecules and excluding the monomers, more than 50% and especially more than 80% of all oligourea molecules have oligomerization degrees of 2 to 16, especially 2 to 8.

3. Method according to claim 1 or 2, wherein the production of the polyurethane and/or polythiourethane compounds occurs in the absence of water, polyurethane catalysts, colorants, stabilizers and/or inflators.

4. Method according to at least one of the preceding claims, wherein the polyurethane and/or polythiourethane compounds are constantly present as liquid, dispersed in liquid, or in dissolved form under the conditions of the reaction.

5. Method according to at least one of the preceding claims, wherein a dispersant is added at least during the depolymerization.

6. Method according to claim 5, wherein the dispersant is a diol or polyol with 2 to 40 carbon atoms.

7. Method according to at least one of the preceding claims, wherein chain build-up and depolymerization are carried out in a single reactor during the reaction.

8. Method according to at least one of the preceding claims, wherein the reaction of chain build-up is carried out at 20 and 120° C. (first reaction temperature) and the depolymerization at greater than 120, preferably greater than 150° C. to 250° C. (second reaction temperature).

9. Method according to at least one of the preceding claims, wherein the oligomeric urea compounds are reacted with one or more reactive groups of fungicide, biocide or herbicide compounds.

10. Method according to at least one of the preceding claims, wherein the primary and/or secondary diamines or polyamines are at least partly fungicide, biocide or herbicide compounds.

11. Method according to at least one of the preceding claims, wherein the second reaction temperature is at least 70° C., especially at least 100° C. higher than the first reaction temperature.

12. Method according to at least one of the preceding claims, wherein the oligourea molecules are present at least partly in particle form and dispersed.

13. Method according to at least one of the preceding claims, wherein the primary or secondary diamines or polyamines, mono-amines, water, urea and/or ammonia are added after formation of the polyurethane and/or polythiourethane compounds and before or during the temperature rise to the second reaction temperature, preferably before the temperature rise to the second reaction temperature.

14. Oligourea dispersions, containing a dispersant and oligourea molecules with 2 to 16, preferably 2 to 8 monomer units, especially on average (excepting monomers) with 2 to 16, preferably on average 2 to 8, monomer units, wherein in particular more than 50%, especially more than 80%, of all oligourea molecules (excepting the monomers) have oligomerization degrees of 2 to 16, especially 2 to 8.

15. Oligourea dispersions according to claim 14, containing a dispersant and oligourea molecules (except monomers) with particle size on average of 4 to 40 nm, preferably 8 to 20 nm.

16. Oligourea dispersions according to at least one of claims 14 to 15, wherein the oligourea molecules have terminal groups chosen from at least one free amino and/or hydroxyl group, carboxyl, or SH group as the terminal group.

17. Oligourea dispersions according to at least one of claims 14 to 15, wherein the dispersant has two free/functional groups per molecule, chosen from the group of —OH, —NH₂ and/or ═NH, preferably entirely or partially —OH.

18. Oligourea dispersions according to at least one of claims 14 to 17, wherein the composition contains
1 to 90 wt. %, or 5 to 50 wt. % of mono- and/or oligourea molecules,
99 to 10 wt. % or 95 to 50 wt. % of dispersant.

19. Oligourea dispersions according to at least one of claims 14 to 18, wherein the composition contains more than 20 wt. %, preferably more than 30 wt. % of oligourea particles.

20. Oligourea dispersions according to at least one of claims 14 to 19, wherein the composition is obtained by one of the methods according to claims 1 to 12.

21. Use of the oligourea dispersions according to at least one of claims 14 to 20 as a fungicide, biocide and/or herbicide, or an ingredient thereof.
